(19) United States
(12) Patent Application Publication
(10) Pub. No.: US 2025/0177204 A1
(43) Pub. Date: Jun. 5, 2025

(54) MARKINGS OR GEOMETRIC GUIDES TO FACILITATE PLACEMENT AND TRAJECTORY OF SURGICAL SUTURES FOR THIN-FILM OCULAR IMPLANTS IN OPEN OR MINIMALLY INVASIVE SURGICAL SITES

(71) Applicant: Avisi Technologies, Inc., Redwood City, CA (US)

(72) Inventors: Shaelynn Fisher, San Jose, CA (US); Jeffrey Lorne Emery, Emerald Hills, CA (US); Georgia Griggs, Philadelphia, PA (US)

(21) Appl. No.: 18/969,792

(22) Filed: Dec. 5, 2024

Related U.S. Application Data
(60) Provisional application No. 63/606,321, filed on Dec. 5, 2023.

(51) Int. Cl. A61F 9/007; A61B 17/04

(52) U.S. Cl. CPC A61F 9/00781; A61B 17/0482; A61F 2250/0097

(57) ABSTRACT

Disclosed herein are thin-film implants or surgical tools including a plurality of surface markings or geometric guides configured to facilitate placement and trajectory of surgical sutures for thin-film ocular implants in open or minimally invasive surgical sites.

### CROSS REFERENCE TO RELATED APPLICATION

The present application claims the benefit of U.S. Provisional Patent Application Ser. No. 63/606,321 filed Dec. 5, 2023, the entire contents of each of which are incorporated by reference herein.

### BACKGROUND

In minimally invasive surgical (MIS) procedures, e.g., endoscopic and laparoscopic surgeries, a surgeon performs diagnostic and therapeutic procedures at a surgical site through a natural body aperture or through one or more small incisions, using instruments specially designed for this purpose. Difficulties encountered by a surgeon in such MIS procedures may include reduced visibility and field of vision, as well as potential orientation difficulties when performing the required manipulations at the surgical site, such as suturing tissue. Suturing procedures can be particularly challenging in MIS procedures. For example, it can be difficult for a surgeon to determine various attributes for the suture being used, such as the number of sutures to be placed, the optimal direction of the suture, and the forces to which the suture is subjected. As a result, use of such sutures adds to the visibility and orientation problems encountered by a surgeon when suturing in a MIS procedure.

Thin-film implants may present unique challenges for ocular fixation techniques. For example, securing ocular implants to tissue is conventionally accomplished by suturing and/or tissue ingrowth. Both options often include having a manufactured hole in the implant for suture placement or for tissue ingrowth. Conventional techniques are a source of large variability in suture anchoring with positioning and the angle of which to install the suture. Currently, there is an unmet need to provide a user with more guidance on the necessary fixation of a thin-film ocular implant, particularly if introduction of holes in the material present manufacturing challenges.

Accordingly, there is a need to provide a device, system and method configured to facilitate placement and trajectory of surgical sutures for thin-film ocular implants in open or minimally invasive surgical sites.

### SUMMARY

Described herein are markings or geometric guides configured to facilitate placement and trajectory of surgical sutures for thin-film ocular implants in open or minimally invasive surgical sites. These markings or geometric guides may be configured to indicate to a user where on a thin-film implant to place a suture to secure the implant onto an eye, as well as what path and/or trajectory the suture should follow.

Among other features, the use of surface marking, implant outline marking, or using an implant-shaped trial of the present disclosure provide suture guides so as to fixate optimally a treatment device to a patient's eye tissue and prevent device migration.

The convenience, reliability, and security of having suture guides unique to a thin-film implant ensure proper fixation and device migration prevention. This approach reduces the variability associated with user chosen suture sites.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTIONS OF THE DRAWINGS

The present disclosure will become more fully understood from the detailed description and the accompanying drawings, wherein:

FIG. 1 is a perspective view of a device according to one embodiment.

FIG. 2 is a close-up view of the device according to section A identified in FIG. 1.

FIG. 3 is a cross-sectional view of the device shown along line III-III in FIG. 2.

FIG. 4 is a perspective view of a device according to another embodiment.

FIG. 5A is a portion of a cross-sectional view of section A of the device shown in FIG. 4 according to one embodiment.

FIG. 5B is a portion of a cross-sectional view of section A of the device shown in FIG. 4 according to one embodiment.

FIG. 5C is a portion of a cross-sectional view of section A of the device shown in FIG. 4 according to one embodiment.

FIG. 6 is a diagram of a treatment device including multiple surface markings, according to an example embodiment of the present disclosure.

FIG. 7 is a schematic diagram of the treatment device of FIG. 6 with suture loops securing the treatment device onto a patient's eye, according to an example embodiment of the present disclosure.

FIG. 8 is a picture showing a user starting a suture bite to place the treatment device of FIG. 6 into a patient's eye, according to an example embodiment of the present disclosure.

FIG. 9 is a diagram of a treatment device having two notches implemented at 4 o'clock and 10 o'clock positions of its main body, according to an example embodiment of the present disclosure.

FIGS. 10 and 11 illustrate an implant body-shaped trial, according to an example embodiment of the present disclosure.

FIGS. 12, 13 and 14 illustrate a treatment device being marked with surgical ink via a trial with under-side protrusions, according to an example embodiment of the present disclosure.

FIGS. 15, 16, and 17 illustrate a treatment device being marked with surgical ink via a trial with under-side protrusions, according to an example embodiment of the present disclosure.

FIGS. 18 and 19 illustrate dimple or surgical ink markings placed directly on the sclera of a patient's eye, according to an example embodiment of the present disclosure.

### DETAILED DESCRIPTION

Various aspects of the present disclosure will be described with reference to the drawings, wherein like reference numerals are used to refer to like elements throughout. In the following description, for purposes of explanation, numerous specific details are set forth in order to promote a thorough understanding of one or more aspects of the present disclosure. It may be evident in some or all instances, however, that any aspects described below can be practiced without adopting the specific design details described below.

It should be appreciated that the device, system and method of the present disclosure may be utilized in any one or more medical or surgical procedures that involve fragile thin-film like implant such as, for example cardiac surgery, anastomosis procedures, non-surgical procedures, endoscopic procedures, non-invasive procedures, invasive procedures, port-access procedures, fluoroscopic procedures, beating heart surgery, vascular surgery, neurosurgery, electrophysiology procedures, diagnostic and therapeutic procedures, ablation procedures, ablation of arrhythmias, endovascular procedures, treatment of one or more organs and/or vessels, cardiograms, pharmacological therapies, drug delivery procedures, delivery of biological agents, gene therapies, cellular therapies, cancer therapies, radiation therapies, genetic, cellular, tissue and/or organ manipulation or transplantation procedures, coronary angioplasty procedures, placement or delivery of coated or uncoated stents, placement of cardiac reinforcement devices, placement of cardiac assistance devices, atherectomy procedures, atherosclerotic plaque manipulation and/or removal procedures, emergency procedures, cosmetic procedures, reconstructive surgical procedures, biopsy procedures, autopsy procedures, surgical training procedures, birthing procedures, congenital repair procedures, and medical procedures that require manipulation and delivery of one or more fragile thin-film like implant into a surgical site.

In one embodiment, as will be described fully below, the present disclosure relates to holding, placement and delivery of a thin-film based ocular implant such as for treatment of glaucoma. A glaucoma drainage implant is a small device (i.e., a thin-film device) placed in an eye of a patient to treat glaucoma. Most glaucoma patients have abnormally high intraocular pressure (IOP) due to the patient's inability to drain excessive aqueous humor from the anterior chamber of the eye through the trabecular meshwork. If not reduced with adequate treatment, high IOP will continuously damage the optic nerve as the disease progresses, leading to loss of vision or even total blindness. During a glaucoma implant surgery, a tiny drainage hole may be made in the sclera of the patient's eye (the white part of the eye). This opening allows fluid to drain out of the eye under the delicate membrane covering the eyeball known as the conjunctiva. Locally applied medications or injections may be used to keep the hole open and a thin-film glaucoma drainage device is positioned on the outside of the eye under the conjunctiva to drain excessive fluid out of the eye and into a place where the capillaries and lymphatic system of the patient reabsorb it back into the body, thereby lowering the intraocular pressure.

To reduce scaring and post-operative patient discomfort, it is desired to make as small as incision of the target implantation site as possible, ideally less than 2 millimeters (“mm”) wide×1 mm tall. However, the treatment device, in many embodiments, has a width between 1 and 10 millimeters, preferably around 2 mm wide, and a height of up to 1 mm, preferably 0.1 mm, to provide to adequate drainage of aqueous humor from an anterior chamber of a patient's eye. Ideally, this means that the delivery tool is minimally wider and taller than the implant itself.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

The description of illustrative embodiments according to principles of the present disclosure is intended to be read in connection with the accompanying drawings, which are to be considered part of the entire written description. In the description of embodiments disclosed herein, any reference to direction or orientation is merely intended for convenience of description and is not intended in any way to limit the scope of the present disclosure. Relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the structure be constructed or operated in a particular orientation unless explicitly indicated as such.

Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the disclosure are illustrated by reference to the exemplified embodiments. Accordingly, the disclosure expressly should not be limited to such exemplary embodiments illustrating some possible non-limiting combination of features that may exist alone or in other combinations of features; the scope of the disclosure being defined by the claims appended hereto.

Unless otherwise specified, all percentages and amounts expressed herein and elsewhere in the specification should be understood to refer to percentages by weight. The amounts given are based on the weight of the material. According to the present application, the term “about” means +/−5% of the reference value. According to the present application, the term “substantially free” means less than about 0.1 wt. % based on the total of the referenced value.

A “subject” herein may be a human or a non-human animal, for example, but not by limitation, rodents such as mice, rats, hamsters, and guinea pigs; rabbits; dogs; cats; sheep; pigs; goats; cattle; horses; and non-human primates such as apes and monkeys, etc.

### Treatment Device Embodiment

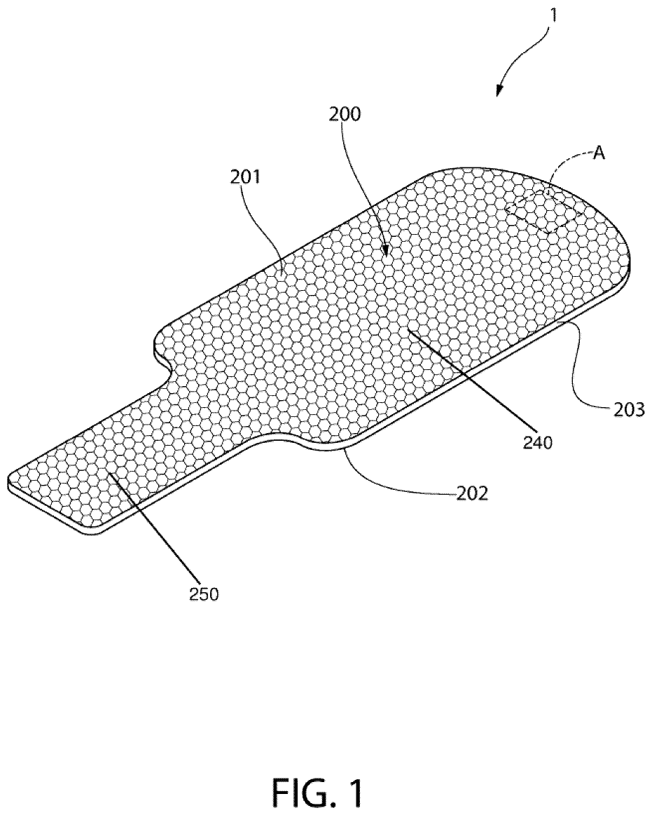

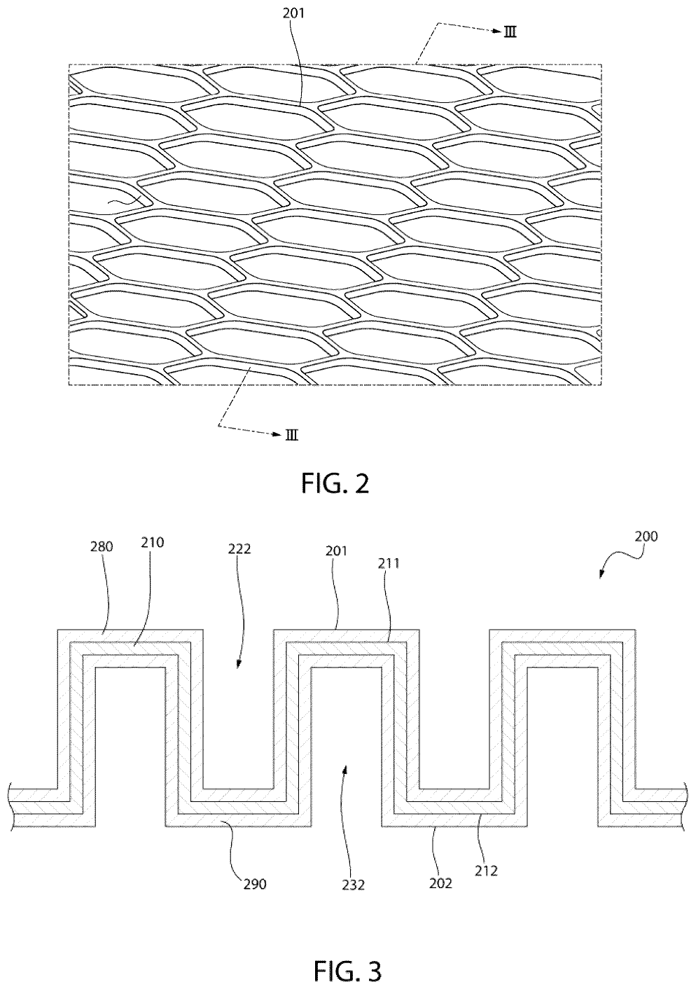

Referring to FIGS. 1-3, a treatment device 1 includes a plate structure 200, or simply plate, having a first major exposed surface 201 opposite a second major exposed surface 202 as well as side surface 203 extending there-between. The plate structure 200 may comprise an extension portion 250 and a main body portion 240.

The plate structure 200 can be formed of any material with appropriate characteristics for implantation and treatment. In some embodiments, the plate structure 200 can be formed of a metal, polymer, ceramic (e.g., aluminum oxide), other composite material, or a combination thereof. Metals can include, but are not limited to aluminum, titanium, zinc, platinum, tantalum, copper, nickel, rhodium, gold, silver, palladium, chromium, iron, indium, ruthenium, osmium, tin, iridium, or combinations, and alloys thereof. In some embodiments, alloys can include steel and nickel titanium such as Nitinol.

Polymers or polymer materials used to form plate structure 200 can include any of the polymers described herein.

Composites such as silicon composites can also be used. In one embodiment, a composite can include silicon nitride (Si₃N₄). The silicon nitride can have any known crystalline structure such as, but not limited to, trigonal α-Si₃N₄, hexagonal β-Si₃N₄, or cubic γ-Si₃N₄.

The plate structure 200, or plate, can have a thickness ranging from about 1 nm to about 1,000 nm, from about 1 nm to about 500 nm, from about 1 nm to about 400 nm, from about 100 nm to about 1,000 nm, from about 200 nm to about 1,000 nm, from about 300 nm to about 1,000 nm, from about 400 nm to about 1,000 nm, from about 1 nm to about 900 nm, from about 1 nm to about 800 nm, from about 1 nm to about 700 nm, from about 1 nm to about 600 nm, from about 300 nm to about 500 nm, from about 300 nm to about 600 nm, from about 400 nm to about 600 nm, from about 200 nm to about 600 nm, from about 200 nm to about 500 nm, or from about 50 nm to about 800 nm.

The plate structure 200 may comprise a multi-directional plate 210 comprising a first major surface 211 opposite a second major surface 212. The multi-directional plate 210 may form a plurality of topographical features (for example, a repeating honeycomb pattern) on each of the first major surface 211 and the second major surface 212. Each of the first and second topographies may independently comprise a plurality of channels 232 and/or a plurality of open-cells 222.

The plurality of channels 232 may be interconnected and can form a network of channels. The channels may be open or closed, allowing fluid to readily enter each channel of plurality of channels 232 and flow through it. The network may comprise intersecting channels in any suitable configuration to best help promote the flow of fluid across the plate structure 200 via the plurality of channels 232. In one embodiment, the channels 232 may be configured to form hexagonal patterns. Once treatment device 1, illustrated in FIG. 1, is implanted, fluid (e.g., aqueous humor) may be driven by a pressure gradient to flow through the channels and across the surface of plate structure 200.

In some embodiments, the channels 232 can include a ribbing pattern. The ribbing pattern and/or the geometry of the channels in the plate can be varied based on different severities of disease (e.g., mild, moderate, or severe glaucoma). In one embodiment, larger or smaller channels can be used to decrease intraocular pressure by different amounts. Changing intraocular pressure by a lower amount can decrease risk of hypotony (a condition that can exist if intraocular pressure is reduced too much) and increase efficacy at lowering pressure to a target level. In some embodiments, a device as described herein with smaller channels can decrease flow and decrease risk of hypotony. Likewise, larger channels can increase flow and allow the device to reduce intraocular pressure to a lower level.

The plate structure 200 may further comprise a first coating 280 applied to the first major surface 211 of the multi-directional plate 210. The first coating 280 may conform to the first topography of the first major surface 211 of the multi-directional plate 210. In other embodiments, the first coating 280 may form a topography that does not conform to the first topography of the first major surface 211 of the multi-directional plate 210.

The first coating 280 may have a thickness ranging from about 0.1 μm to about 10 μm or about 0.1 μm to about 2 μm—including all thickness and sub-ranges there-between. In one embodiment, the thickness is between about 0.4 μm (400 nm) and 0.6 μm (600 nm). In one embodiment, the thickness is about 0.4 μm (400 nm). In other embodiments, the thickness is between about 1 μm and about 5 μm, between about 1 μm and about 3 μm, between about 2 μm and about 5 μm, or between about 2 μm and about 4 μm. In one embodiment, the thickness is about 2 μm.

The plate structure 200 may further comprise a second coating 290 applied to the second major surface 212 of the multi-directional plate 210. The second coating 290 may conform to the plurality of surface features on the second major surface 212 of the multi-directional plate 210. In other embodiments, the second coating 290 may form a topography that does not conform to the second topography of the second major surface 212 of the multi-directional plate 210.

The second coating 290 may have a thickness ranging from about 0.1 μm to about 10 μm or about 0.1 μm to about 1 μm—including all thickness and sub-ranges there-between. In one embodiment, the thickness is between about 0.4 μm (400 nm) and 0.6 μm (600 nm). In one embodiment, the thickness is about 0.4 μm (400 nm). In other embodiments, the thickness is between about 1 μm and about 5 μm, between about 1 μm and about 3 μm, between about 2 μm and about 5 μm, or between about 2 μm and about 4 μm. In one embodiment, the thickness is about 2 μm.

In some embodiments, the plate structure 200 may comprise only the first coating 280—i.e., no second coating. In other embodiments, the plate structure 200 may comprise only the second coating 290—i.e., no first coating. In other embodiments, the plate structure 200 may comprise the first coating 280 and the second coating 290, whereby the first and second coatings overlap to fully encapsulate the multi-directional plate 210. In such embodiments, the side surface 203 of the plate structure 200 may comprise at least one of the first coating 280 and the second coating 290.

In some embodiments, the first and second coating, and any edge coating, can be thicker than the plate itself. In some embodiments, the coating thickness can be one, two or three orders of magnitude thicker than the plate structure. However, in other embodiments, the plate can be thicker than each coating or the additive thickness of the two coatings.

Coatings described herein can be applied by any suitable deposition method, such as but not limited to, physical vapor deposition, chemical vapor deposition, atomic layer deposition, spray coating, spin coating, self-assembly, dip coating, or brushing.

The first coating 280 may be applied to the first major surface 211 by any suitable deposition method. In a non-limiting example, the first coating 280 may be applied to the first major surface 211 by chemical vapor deposition, physical vapor deposition, or plasma-enhanced chemical vapor deposition. In another non-limiting example, the first coating 280 may be applied to the first major surface 211 by atomic layer deposition. In another non-limiting example, the first coating 280 may be applied to the first major surface 211 by spray coating. In another non-limiting example, the first coating 280 may be applied to the first major surface 211 by dip coating. In another non-limiting example, the first coating 280 may be applied to the first major surface 211 by brushing.

The second coating 290 may be applied to the second major surface 212 by any suitable deposition method. In a non-limiting example, the second coating 290 may be applied to the second major surface 212 by chemical vapor deposition, physical vapor deposition, or plasma-enhanced chemical vapor deposition. In another non-limiting example, the second coating 290 may be applied to the second major surface 212 by atomic layer deposition. In another non-limiting example, the second coating 290 may be applied to the second major surface 212 by spray coating. In another non-limiting example, the second coating 290 may be applied to the second major surface 212 by dip coating. In another non-limiting example, the second coating 290 may be applied to the second major surface 212 by brushing.

The first coating 280 may be the same as the second coating 290. The first coating 280 and the second coating 290 may be different. The first coating 280 may be hydrophilic. The first coating 280 may be hydrophobic. The first coating 280 may be lipophilic. The first coating 280 may be lipophobic. The second coating 290 may be hydrophilic. The second coating 290 may be hydrophobic. The second coating 290 may be lipophilic. The second coating 290 may be lipophobic. Each of the first and second coatings 280, 290 may independently be continuous. Each of the first and second coatings 280, 290 may independently be discontinuous. In some embodiments, the first and second coatings 280, 290 may both be hydrophobic. In some embodiments, the first and second coatings 280, 290 may both be hydrophilic. In some embodiments, the first and second coatings 280, 290 may both be lipophilic or lipophobic.

The first coating 280 may be organic. The first coating 280 may be inorganic. The second coating 290 may be organic. The second coating 290 may be inorganic.

In some embodiments, the first coating 280 is hydrophilic and the second coating 290 is hydrophobic. In some embodiments, the first coating 280 is hydrophilic and the second coating 290 is hydrophilic. Having at least one of the first and/or second coating 280, 290 be hydrophobic may help prevent the treatment device 1 from inadvertently sticking to tissue during implantation.

In some embodiments, a purpose of a first and/or second coating is to increase the toughness of the device. Also, a first and/or second coating can increase biocompatibility of the device and/or decrease scarring by decreasing tissue and/or fibroblast adhesion. In some embodiments, the coatings described herein are hydrophobic and decrease tissue adhesion. In some embodiments, tissue adhesion can be reduced by greater than about 10%, greater than about 60%, greater than about 70%, greater than about 80%, greater than about 90%, greater than about 95%, greater than about 96%, greater than about 97%, greater than about 98%, or greater than about 99% when compared to an uncoated plate.

In a non-limiting embodiment, the first and/or second coating may comprise a polymer, such as a parylene polymer (poly(para-xylylene)) or a derivative thereof. In other embodiments, the first and/or second coating can include aluminum oxide, a biocompatible film, a porous coating, or a lubricious coating. In one embodiment, the parylene polymer is a chlorine modified poly(para-xylylene), or a fluorine modified poly(para-xylylene). In one embodiment, the parylene polymer can be parylene C, parylene D, parylene N, a derivative thereof or a combination thereof. In other embodiments, the first and/or second coating can include aluminum oxide.

In other embodiments, other polymer(s) can be used in addition to, in combination with, or instead of a parylene polymer and/or aluminum oxide. In some embodiments, other polymeric materials can include, but are not limited to rubber, synthetic rubber, silicone polymers, thermoplastics, thermosets, polyolefins, polyisobutylene, acrylic polymers, ethylene-co-vinylacetate, polybutylmethacrylate, vinyl halide polymers (for example, polyvinyl chloride), polyvinyl ethers (for example, polyvinyl methyl ether), polyvinylidene halides, polyacrylonitrile, polyvinyl ketones, polyvinyl aromatics, polyvinyl esters, acrylonitrile-styrene copolymers, ABS resins, ethylene-vinyl acetate copolymers, polyamides (for example, Nylon 66 and polycaprolactam), alkyd resins, polycarbonates, polyoxymethylenes, polyimides, polyethers, epoxy resins, polyurethanes, rayon, cellulose, cellulose acetate, cellulose butyrate, cellulose acetate butyrate, cellophane, cellulose nitrate, cellulose propionate, cellulose ethers, carboxymethyl cellulose, polytetrafluororethylene (for example, Teflon), poly(ether-ether-ketone), poly lactides such as PLA, PLGA, PLLA, derivatives thereof, or combinations thereof.

The resulting treatment device 1 may comprise the first plurality of channels 222 present on the first exposed major surface 201 of the plate structure 200, wherein the first plurality of channels 222 are hydrophilic due to the presence of the first coating 280. The resulting treatment device 1 may comprise the second plurality of channels 232 present on the second exposed major surface 202 of the plate structure 200, wherein the second plurality of channels 232 are hydrophilic due to the presence of the second coating 290. As discussed, the hydrophilic channels may promote fluid flow through the channels after the treatment device 1 has been implanted into a subject's eye.

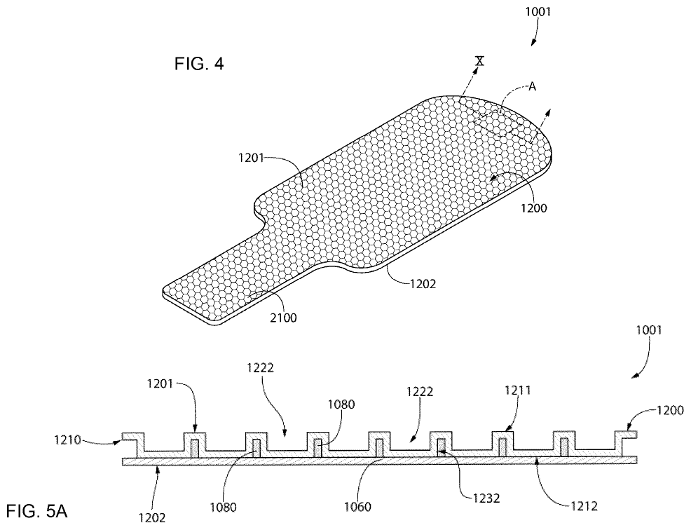

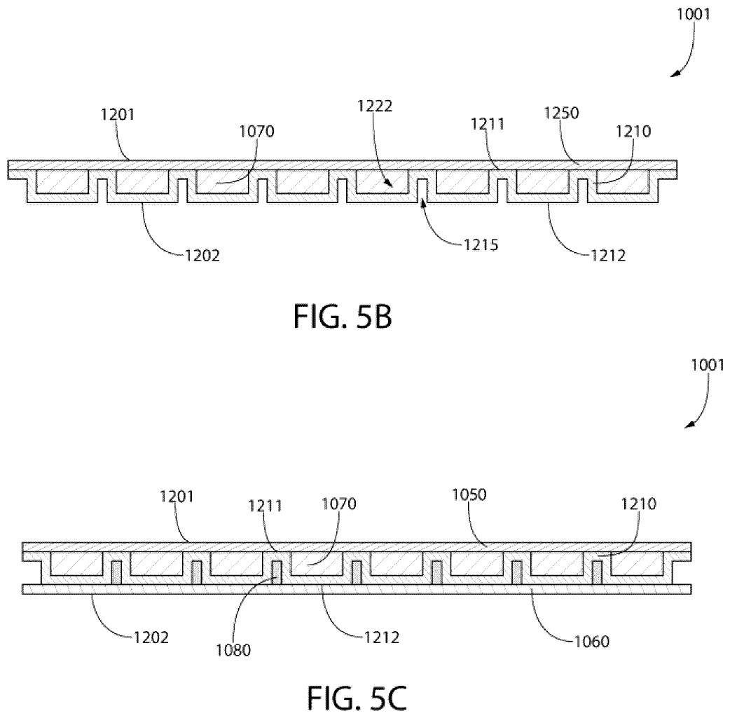

Referring to FIGS. 4, 5A, 5B, and 5C, generally, a treatment device 1001 is illustrated in accordance with another embodiment. The treatment device 1001 is similar to the treatment device 1 except as described herein below. The description of the treatment device 1 above generally applies to the treatment device 1001 described below except with regard to the differences specifically noted below. A similar numbering scheme will be used for the treatment device 1001 as with the treatment device 1 except that a “1000” series of numbering will be used.

The treatment device 1001 comprises a plate structure 1200 having a first exposed major surface 1201 that is opposite a second exposed major surface 1202. The plate structure 1200 may comprise a multi-directional plate 1210 comprising a first major surface 1211 opposite a second major surface 1212. The multi-directional plate 1210 may form a plurality of topographical features (for example, a repeating honeycomb pattern) on each of the first major surface 1211 and the second major surface 1212. Each of the first and second topographies may independently comprise a plurality of channels 1232 and/or a plurality of open-cells 1222.

Referring now to FIG. 5B, the plate structure 1200 may comprise a first drug-treatment delivery component 1070 present in the open voids created by the first topography formed by the first exposed surface 1211 of the multi-directional plate 1210. Specifically, the first delivery component 1070 may be present in the open voids created by the open-cells 1222 of first topography formed by the first major surface 1211 of the multi-directional plate 1210.

The first drug-treatment delivery component 1070 may comprise one or more active agents such as, but not limited to therapeutic and/or pharmacological components. The first drug-treatment delivery component 1070 may occupy some, all, or substantially all of the free volume present in the open-cells 1222 formed by the first topography.

In other embodiments, active agents can include any compound or drug having a therapeutic effect in a subject. Non limiting active agents include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, steroids, proteasome inhibitors, antibiotics, anti-inflammatories, anti-sense nucleotides, transforming nucleic acids, messenger ribonucleic acids, IOP lowering drugs, prostaglandins, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, cells, stem cells, liposomes, anti-metabolites such as mitomycin-C, combinations thereof, prodrugs thereof, pharmaceutical salts thereof, derivatives thereof, and the like.

The treatment device 1001 may further comprise a first coating 1050 applied to a first major surface 1211 of the multi-directional plate 1210. The first coating 1050 may cover both a first major surface 1211 of the multi-directional plate 1210 as well as a first drug-treatment delivery component 1070 that is present in the open-cells 1222 formed into the first major surface 1211 of the multi-directional plate 1210. The first coating 1050 may be in the form of a continuous film. The first coating 1050 may be flat. In other embodiments, the first coating 1050 may be conformal to the underlying pattern formed by the multi-directional plate 1210 and the first delivery component 1070.

Referring now to FIG. 5A, the plate structure 1200 may comprise a second drug-treatment delivery component 1080 present in the open voids created by the second topography formed by the second exposed surface 1212 of the multi-directional plate 1210. Specifically, the second delivery component 1080 may be present in the open voids created by the open-channels 1232 of the second topography formed by the second major surface 1212 of the multi-directional plate 1210. The second delivery component 1080 may be the same or different from the first delivery component 1070.

The second drug-treatment delivery component 1080 may comprise one or more therapeutic and/or pharmacological components-including but not limited to anti-inflammatory agents, steroids, antibiotics, analgesics. The second delivery component 1080 may occupy some, all, or substantially all of the free volume present in the channels 1232 formed by the first topography.

The treatment device 1001 may further comprise a second coating 1060 applied to a second major surface 1212 of the multi-directional plate 1210. The second coating 1060 may cover both the second major surface 1212 of the multi-directional plate 1210 as well as the second delivery component 1080 that is present in the open-channels 1232 formed into the second major surface 1212 of the multi-directional plate 1210. The second coating 1060 may be in the form of a continuous film. The second coating 1060 may be flat. In other embodiments, the second coating 1060 may be conformal to the underlying pattern formed by the multi-directional plate 1210 and the second delivery component 1080.

The second coating 1060 may be the same or different than the first coating 1050. For each of the first and the second coatings 1050, 1060, the resulting film may be formed from a slow-release material that dissolves slowly after exposure to aqueous humor or other biological fluids, thereby releasing the first delivery component 1070 from the channels 1232 of the treatment device 1001 after it has been implanted into a subject.

Referring now to FIG. 5C, in other embodiments, the treatment device 1001 may comprise both the first and the second drug-treatment delivery components 1070, 1080, as well as the first and the second coatings 1050, 1060 to encapsulate the first and second delivery components 1070, 1080.

In other embodiments, the plate structure 1200 may comprise at least one of the first coating 1050 and/or the second coating 1060 without the presence of the first and/or second delivery components 1070, 1080. In such embodiments, the first coating 1050 and/or the second coating 1060 may form a film that covers the open cells 1222 and/or the open channels 1232 created by the multi-directional plate.

The presence of the films resulting from the first and/or the second coating 1050, 1060 may enhance the overall strength of the resulting treatment device. Specifically, layered structure(s) of the films formed by the first and second coatings 1050, 1060, which are bonded to the first and second major surfaces 1211, 1212 of the multi-directional plate 1210, provide added mechanical integrity to the resulting treatment device.

Beyond achieving the baseline flexibility to conform to curvature of the eye, the addition of the first and/or second coatings 1050, 1060 may provide a mechanism that allows the overall treatment device to match the elastic modulus of surrounding tissues (e.g., conjunctival and scleral tissues) to maximize biocompatibility or biointegration. Findings in brain implant research confirm that the flexibility of implants in soft tissue improves compliance of the implant with microscale movements of surrounding tissue and reduces tissue displacement and trauma as well as facilitates implantation of the treatment device.

### Surface Markings

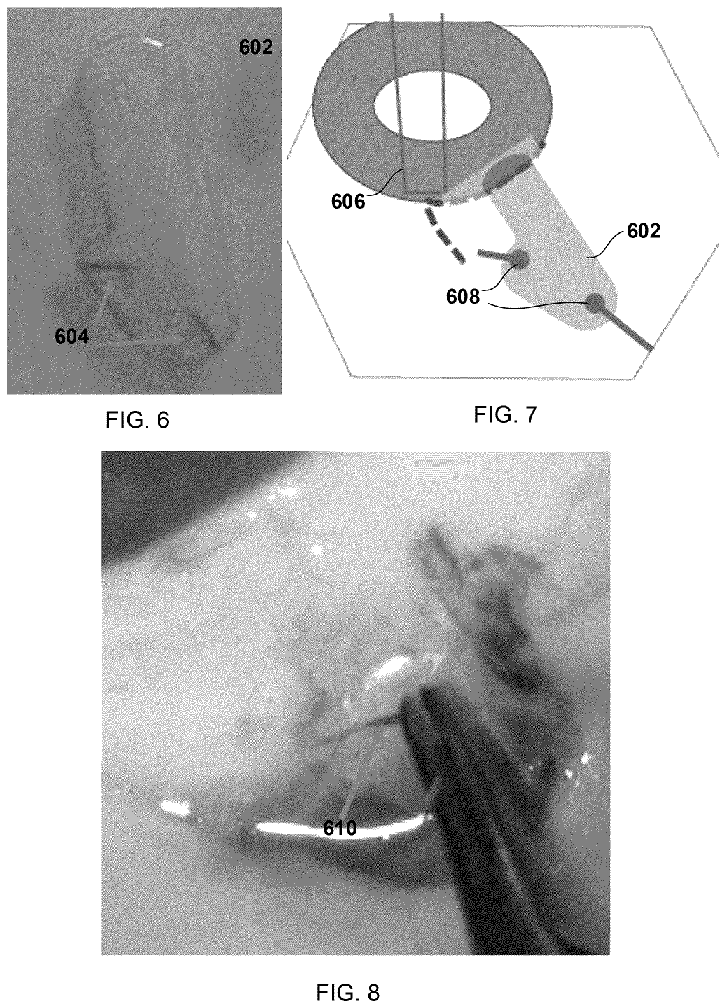

Referring to FIGS. 6, 7, and 8, a thin-film implant 602, which is similar to the treatment device 1 of FIGS. 1-3 or the treatment device 1001 of FIGS. 4, 5A, 5B, and 5C, may comprise a plurality of surface markings 604 for providing placement and trajectory of surgical sutures, in accordance with aspects of the present disclosure. FIG. 7 is a schematic diagram of the thin-film implant 602 of FIG. 6 with suture loops 608 securing the implant 602 onto a patient's eye. Suture loops 608 follow the suture surface markings 604, where the dot 608 indicates the piercing location of a suture needle (not shown), and the suture thread loop is aligned with the laser marks. Surface markings such as surface modification on the thin-film implant 602 via laser or any suitable techniques can identify the origin of the suture anchor (i.e., where to pierce the thin-film implant 602 with a suture needle), as well as the trajectory for how to orient a path of the suture loop 608. All sutures produce vector forces that act in various directions as the suture is tightened. Generally, the vector forces extend in three different directions: perpendicular to the wound surface, parallel to the wound margin, and perpendicular to the tissue surface. If a suture is placed perpendicular to the wound surface, the force vectors cause compression in a line where the suture plane intersects with the wound surface. However, if the suture is placed obliquely, the compression vector force is an area on the wound surface; therefore, a lateral shift of the wound is produced. This shift is also the result of the vector force that is parallel to the wound margin. This force is not generated when the interrupted sutures are placed perpendicular to the wound. In continuous sutures, the shifting vectors of the bridging segments of the suture can serve to neutralize the shifting forces generated by each suture bite. The third vector component, perpendicular to the tissue surface, results in two forces in opposite direction in the simple interrupted suture. The first component results in eversion of the wound edge, and the second portion of the suture generates a force resulting in inversion of the wound edge. In a simple interrupted suture, these forces cancel each other out, and they are in opposite directions. Continuous sutures produce both inverting and everting forces that are cancelled out if the loops are placed very close together, otherwise, significant irregularities of the tissue surface result. The effects of compressing vectors are maximal in the suture plane and diminish farther away from the suture. Each interrupted suture generates a zone of compression. The compressive effect is maximal in the plane between the point of suture entry and suture exit and falls off laterally. The action of the suture can be described in terms of force triangles extending laterally from the suture. The width of these compression zones depends on the length of the suture bites and the degree of suture tension after the suture is tightened. Adequate wound closure is achieved when the zones of compression of each interrupted suture overlap.

The surface markings 604 of the present disclosure can be critical for identifying the proper vectors of tension forces created by the suture anchors that are required for long-term stability of the thin-film implant position to prevent migration and associated safety and efficacy issues.

In certain embodiments, the plurality of surface markings 604 of the thin-film implant 602 may be generated via e.g., an excimer laser or any suitable techniques, to permanently mark the surface of the thin-film implant 602 at selected locations. A user may start a suture bite at the start of the line 610 as shown in FIG. 8 and follow the markings 604 to the implant perimeter with the exit and tying of the suture knot. The markings 604 provides a starting point and a vector for suture placement to ensure proper fixation of the thin-film implant 602 and prevent the implant's movements in or out of the anterior chamber. Surface markings may include text or shapes such as lines, arrows, arrowheads, curves, or any similar permutation which conveys to the surgeon the starting point for the suture bite and optimal trajectory for the suture orientation.

### Geometric Guides

In accordance with other aspects, the present disclosure relates to geometric guide(s) achieved with edge features (i.e., notches) as part of a thin-film implant outline.

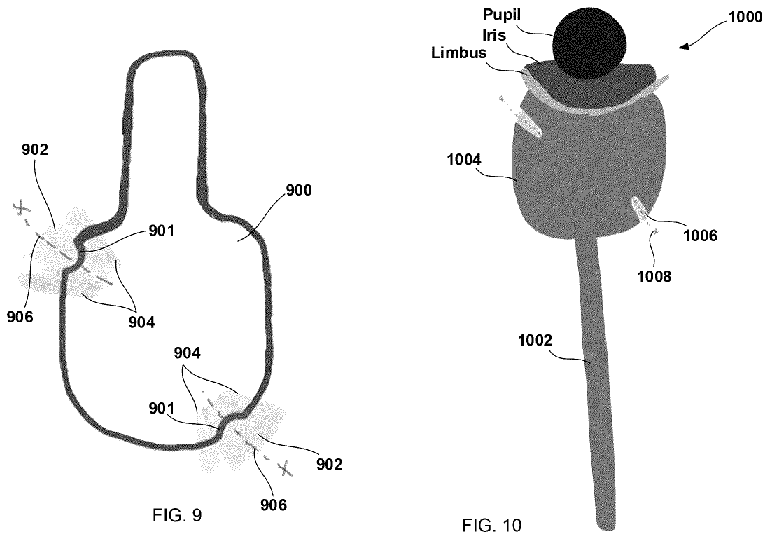

FIG. 9 illustrates a thin-film implant 900 having two notches 901 implemented at 4 o'clock and 10 o'clock positions of its main body, according to a preferred embodiment. Region 902 is in the location of each notch 901, region 904 surrounds each region 902 and is outside of each notch 901, and dashed line 906 is the path of a suture loop. Implementing selected features such as a notch on a thin-film implant may function as a visual indicator to a user where to start a suture loop and then finish it. In another embodiment, multiple notches may be included for guiding sutures as well. For example, a user may start a suture bite near a curved portion (e.g., a round curve) of each notch 901 and follow the notch opening for the exit and tying of the suture knot. This provides a starting point and a vector for suture placement to ensure proper fixation of a thin-film implant as well as aiming to prevent the implant's movements in or out of the anterior chamber.

It should be appreciated that there may be more than one surface marking or geometric guide depending on the needs of the thin-film implant design and fixation needs. For example, larger designs of certain treatment device may, although not necessarily, require more suture anchors to ensure that the device is stable and remains flat on the tissue.

With respect to marking configurations, multiple designs may be utilized including but not limited to straight lines, dashed lines, end of lines marked with a suitable indicators such as X, O, arrows, or other figures or symbols to facilitate interpretation of proper origin and/or trajectory of the suture.

Further, notching configurations may include but not limited to a semicircular, triangular, rectangular, or other polygon, or a combination thereof. In an embodiment, radius corners may be implemented on each notch to reduce stress concentration.

Additional alternate embodiments may include a combination of markings and notching described above.

### Trial Marking System

In certain situations, implementing permanent surface marking or notching on a thin-film implant may undermine the mechanical integrity of the treatment device. Marking systems implemented by the user with surgical ink during procedural installation may offer advantages such as requiring no implant manufacturing modifications, and having no adverse impact on the mechanical integrity of the treatment device. Moreover, this type of marking may be temporary in nature and dissipate quickly with time.

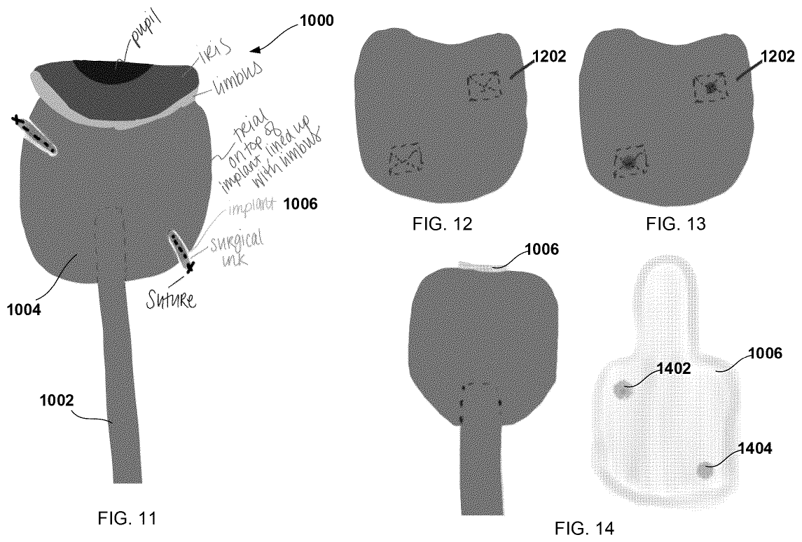

Referring to FIGS. 10 and 11, in accordance with aspects of the present disclosure, a trial marking system 1000 may include an implant body-shaped trial (also known as surgical template) which is a common surgical tool that will show adequate surgical site preparation (i.e., pocket creation). In one embodiment, the implant body-shaped trial may include an elongated handle 1002 connected to a main body 1004 which is overlaid on top of a thin-film implant 1006 lined up with limbus of a patient's eye. In an aspect, the trial may be configured to function as a guide for suture placement. To show the trajectory of suture, the trial may have cut outs (openings) (e.g., at 4 o'clock and 10 o'clock positions) only where sutures 1008 should be placed and directed. Specifically, the trial may lay on top of the body of the thin-film implant 1006 to show where to throw and angle the suture. The cut outs (openings) of the trial may be filled with surgical ink by a user to temporarily mark the thin-film implant 1006 via a non-damaging manner.

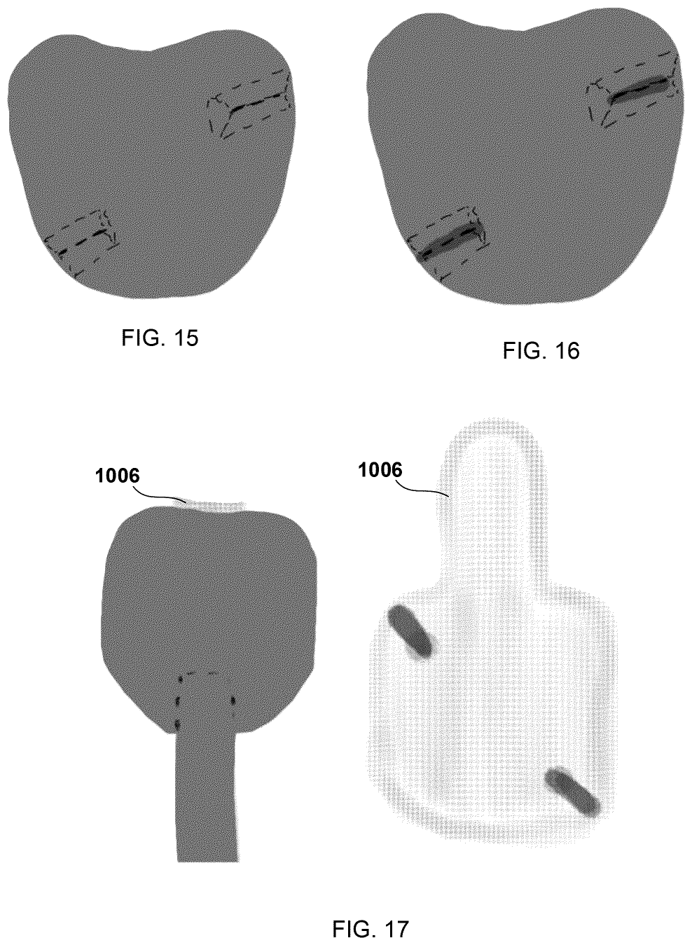

According to some embodiments, as shown in FIGS. 12, 13 and 14, the user may mark the thin-film implant with surgical ink via a trial with under-side protrusions. For example, multiple protrusions 1202 may be wetted or primed with surgical ink and stamped on the thin-film implant 1006 to leave ink markings 1402, 1404 on the thin-film implant 1006. It should be appreciated that different configurations on these protrusions may be implemented based on implant configuration and surgical needs, as shown in FIGS. 15, 16, and 17.

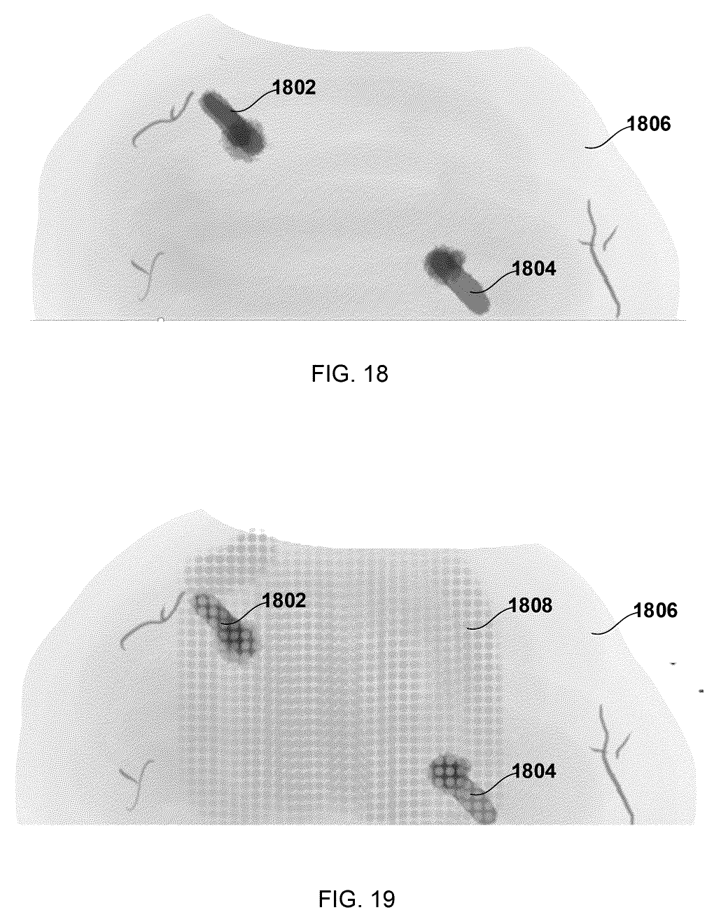

Alternatively, as shown in FIGS. 18 and 19, a dimple with surgical ink markings 1802, 1804 may be placed directly on the sclera 1806 of a patient's eye, rather than on a thin-film implant 1808, and the ink markings 1802, 1804 may become visible due to the transparency of the thin-film implant 1808, thereby providing suture guides to a user.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

Unless otherwise indicated, all numbers expressing quantities and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of this disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific example embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Example embodiments of the disclosure so claimed are inherently or expressly described and enabled herein.

In closing, it is to be understood that the embodiments disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

1. A thin-film implant that lowers intraocular pressure, the thin-film implant comprising:
a first surface opposite a second surface;
a plurality of topographical features on each of the first and second surfaces to control aqueous humor flow in a patient's eye for regulating the intraocular pressure; and
a plurality of surface markings implemented on the first surface and configured to identify an origin of a suture anchor of the thin-film implant and a trajectory of surgical sutures for securing the thin-film implant onto the patient's eye.

2. The thin-film implant of claim 1, wherein the plurality of surface markings are implemented on a surface of the thin-film implant via an industrial laser such as excimer-based.

3. The thin-film implant of claim 1, wherein the plurality of surface markings include a plurality of straight lines.

4. The thin-film implant of claim 1, wherein the plurality of surface markings include a plurality of dashed lines.

5. The thin-film implant of claim 1, wherein the plurality of surface markings include a plurality of lines with at least one end indicator, wherein the at least one indicator includes at least one of a letter, an arrow, a figure or a symbol to facilitate an interpretation of the trajectory of surgical sutures.

6. A thin-film implant that lowers intraocular pressure, the thin-film implant comprising:
a first surface opposite a second surface;
a plurality of topographical features on each of the first and second surfaces to control aqueous humor flow in a patient's eye for regulating the intraocular pressure; and
a plurality of geometric guides implemented on an outer edge of the thin-film implant and configured to identify an origin and/or trajectory of surgical sutures for securing the thin-film implant onto the patient's eye.

7. The thin-film implant of claim 6, wherein the plurality of geometric guides include edges features implemented on an outline of the thin-film implant.

8. The thin-film implant of claim 6, wherein the edges features include a plurality of notches positioned on an outer edge of the thin-film implant.

9. The thin-film implant of claim 8, wherein the plurality of notches are implemented at a 4 o'clock and 10 o'clock positions of a main body of the thin-film implant.

10. The thin-film implant of claim 8, wherein the plurality of notches indicate which side of the thin-film implant should be facing up.

11. The thin-film implant of claim 8, wherein each of the plurality of notches has a semicircular shape.

12. The thin-film implant of claim 8, wherein each of the plurality of notches has a triangular, rectangular, or polygonal shape.

13. The thin-film implant of claim 8, wherein each of the plurality of notches has a shape that is a combination of a semicircular, triangular, rectangular, or polygonal shape.

14. The thin-film implant of claim 8, wherein at least one of the plurality of notches has a radius corner.

15. A surgical tool, comprising:
an implant body-shaped trial overlaid on top of a thin-film implant that lowers intraocular pressure, wherein the thin-film implant includes a first surface opposite a second surface, a plurality of topographical features on each of the first and second surfaces to control aqueous humor flow in a patient's eye for regulating the intraocular pressure; and
one or more openings implemented on a main body of the implant body-shaped trial to indicate a placement and direction of a suture for securing the thin-film implant onto the patient's eye.

16. The surgical tool of claim 15, wherein the one or more openings include a plurality of notches positioned on an outer edge of the main body of the implant body-shaped trial.

17. The surgical tool of claim 16, wherein the plurality of notches are implemented at a 4 o'clock and 10 o'clock positions of the main body of the implant body-shaped trial.

18. The surgical tool of claim 16, wherein the plurality of notches are filled with surgical ink to temporarily mark the thin-film implant.

19. A surgical tool, comprising:
an implant body-shaped trial overlaid on top of a thin-film implant that lowers intraocular pressure, wherein the thin-film implant includes a first surface opposite a second surface, a plurality of topographical features on each of the first and second surfaces to control aqueous humor flow in a patient's eye for regulating the intraocular pressure; and
one or more protrusions implemented on a main body of the implant body-shaped trial, wherein the one or more protrusions are primed with surgical ink to temporarily mark the thin-film implant for securing the thin-film implant onto the patient's eye.

20. A surgical tool, comprising:
an implant body-shaped trial configured to provide suture guides to fixate a thin-film implant to eye tissue and prevent a device migration of the thin-film implant, wherein the thin-film implant includes a first surface opposite a second surface, a plurality of topographical features on each of the first and second surfaces to control aqueous humor flow in a patient's eye for regulating the intraocular pressure.

21. The surgical tool of claim 20. wherein the implant body-shaped trial includes one or more protrusions implemented on a main body of the implant body-shaped trial, wherein the one or more protrusions are primed with surgical ink to temporarily mark the sclera of the eye tissue.
